(19) United States
(12) Patent Application Publication
(10) Pub. No.: US 2026/0249009 A1
(43) Pub. Date: Aug. 27, 2026

(54) DUAL CHAMBER HYDRAULIC AUTO-INJECTOR

(71) Applicant: RX BANDZ, INC.

(72) Inventors: Stephen Harhen, Rumson, NJ (US); Grant Miller, Washington, NJ (US)

(21) Appl. No.: 19/545,511

(22) Filed: Feb. 20, 2026

Related U.S. Application Data
(60) Provisional application No. 63/761,317, filed on Feb. 21, 2025.

(51) Int. Cl. A61M 5/19; A61M 5/20; A61M 5/315; A61M 5/32

(52) U.S. Cl. CPC A61M 5/19; A61M 5/2053; A61M 5/2066; A61M 5/3158; A61M 5/31596; A61M 5/3202; A61M 5/322; A61M 2202/0007; A61M 2202/04

(57) ABSTRACT

An injector for a first therapeutic agent and a second therapeutic agent is provided. The injector includes a housing, a drive mechanism, an actuator, a first chamber assembly, and a second chamber assembly. The housing can have a longitudinal axis and a drive mechanism can include a barrier movable along the longitudinal axis of the housing. The first chamber assembly and the second chamber assembly can include a container, a plunger, and a channel. The first container can house the first therapeutic agent. The first channel can be positioned at a first height within the first chamber. The second channel can be positioned at a second height within the second chamber to allow for distribution of the first therapeutic agent and the second therapeutic agent separately.

### CROSS-REFERENCE TO RELATED APPLICATIONS

This application claims the benefit of U.S. Provisional Application No. 63/761,317, filed on February 21, 2025. The entire disclosure of the above application is incorporated herein by reference.

### FIELD

The present technology relates to fluidic throttle control for delivery of a therapeutic substance in a needle-based delivery system.

### INTRODUCTION

This section provides background information related to the present disclosure which is not necessarily prior art.

An injector can be used to administer a therapeutic agent under emergency conditions, such as, for example, administering epinephrine to counteract the effects of a severe allergic reaction (e.g., anaphylaxis) or naloxone for opioid overdose. An injector can also be used in administering a therapy to treat diseases, such as, for example, anti-arrhythmic medication and selective thrombolytic agents during a heart attack. One such delivery device is an auto-injector, which offers an alternative to a manually operated syringe, as the auto-injector can be used to allow a patient to self-administer a medication in a predetermined dose.

The auto-injector can provide a method of delivery for many therapeutics due to the ease of use by both trained and untrained professionals. Additionally, the speed of delivery and dose accuracy can be predetermined for controlled administration. With an auto-injector, a small diameter needle can be desirable to reduce pain associated with needle insertion. However, the small inner diameter and length of the needle acts to create a restricted fluid flow, which can present an issue in delivering a predetermined amount of fluid in a predetermined time. This issue becomes more of a problem as the needle inner diameter size decreases. Slowdown or stalling of fluid in the needle can be exacerbated in delivery of viscous, shear sensitive or non-Newtonian pharmaceutical therapies, such as proteins and monoclonal antibodies.

To overcome the restrictive inner diameter of the needle, higher pressure can be applied. Such higher pressure, however, can increase the risk of container rupture or stack clogging in the needle. The high initial force can result in blockage or an exponential increase in apparent viscosity of the fluid due to the needle acting as a block in the system, which can result in undelivered or, in some cases, a damaged therapeutic in the fluid due to the shear force experienced by the fluid. Compared to other therapies, rheologically challenging therapies can present more challenges. For example, such therapies can require higher forces to extrude the therapeutic agent through a small diameter needle and are also more likely to clog in the needle. As an alternative, a larger diameter needle in conjunction with a less powerful energy system can be used, but such a needle can cause more patient discomfort.

To provide a pressure high enough to overcome stalling in a small needle, a powerful single energy force, such as a spring, can be used to deploy the therapeutic agent. The force generated by a spring energy source, however, can exacerbate the risk of system failure. Specifically, the high speed and impact force experienced during spring deployment in auto-injector activation can result in mechanical damage to the surface of a container, which can fracture, as the container can come into contact with the interior components and/or interior walls of the auto-injector.

The auto-injector can further be limited to delivering a single therapeutic agent per device, creating challenges in scenarios requiring multiple therapeutic agents for effective treatment. Additionally, the risk of therapeutic agent interaction or cross-contamination during storage or at the injection site, delayed administration in an emergency, and increased complexity for users managing multiple injections can occur when using multiple single therapeutic agent auto-injectors. The challenge of delivering multiple therapeutic agents without interaction during storage or at the injection site can be addressed through various methods, each with significant limitations. One approach is the use of separate devices for each therapeutic agent, requiring a patient to manage and carry multiple injectors. While ensuring therapeutic agent separation, using multiple devices increases the complexity of emergency protocol and adds to manufacturing, packaging, and logistical costs.

Accordingly, there is a continuing need for an injector system with a dual chamber that enables administration of two distinct therapeutic agents from separate therapeutic agent containers without risk of chemical interactions during storage.

### SUMMARY

In concordance with the instant disclosure, an injector system with a dual chamber that enables administration of two distinct therapeutic agents in separate primary therapeutic agent containers without risk of chemical interactions during storage, has surprisingly been discovered. The present technology includes articles of manufacture, systems, and processes that relate to a dual chamber hydraulic auto-injector system for delivering multiple therapeutic agents.

In certain embodiments, an injector for a first therapeutic agent and a second therapeutic agent is provided. The injector includes a housing, a drive mechanism, an actuator, a first chamber assembly, and a second chamber assembly. The housing can define a first chamber and a second chamber. The housing can have a longitudinal axis. The drive mechanism can include hydraulic fluid and a barrier configured to rupture under hydraulic pressure at a predetermined pressure. The actuator can be operatively coupled to the drive mechanism. The first chamber assembly can be disposed within the first chamber and can include a first container, a first plunger, and a first channel. The first container can be disposed within the first chamber and can be configured to house the first therapeutic agent. The first plunger can be operatively associated with the first container. The first channel can be coupled to the housing at a first height within the first chamber and can be configured to direct hydraulic fluid into the first chamber. The second chamber assembly can be disposed within the second chamber and can include a second container, a second plunger, and a second channel. The second container can be disposed within the second chamber and can be configured to house the second therapeutic agent. The second plunger can be operatively associated with the second container. The second channel can be coupled to the housing at a second height within the second chamber and can be configured to direct hydraulic fluid into the second chamber. The second height can be different from the first height. The first channel can be positioned closer to the drive mechanism than the second channel along the longitudinal axis, and the barrier can initially block a hydraulic fluid pathway to the second channel. Upon a first actuation of the actuator, hydraulic fluid can flow through the first channel into the first chamber to drive the first container and cause delivery of the first therapeutic agent from the first container. Upon a second actuation of the actuator, the barrier can rupture to open the hydraulic fluid pathway to the second channel, allowing hydraulic fluid to flow through the second channel into the second chamber to drive the second container and cause delivery of the second therapeutic agent from the second container.

In certain embodiments, a method for dispensing a first therapeutic agent and a second therapeutic agent is provided. The method can include a step of providing an injector as described herein. The method can include activating the actuator, whereby the drive mechanism moves hydraulic fluid through the first channel to move the first plunger to contact and laterally move the first container within the first chamber such that a first needle is placed in fluid communication the first container and dispenses the first therapeutic agent.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations and are not intended to limit the scope of the present disclosure.

FIG. 1 is a perspective view of an injector for a first therapeutic agent and a second therapeutic agent;

FIG. 2 is a front elevational view of the injector with a housing shown in phantom;

FIG. 3 is a rear elevational view of the injector with the housing shown in phantom;

FIG. 4A is a perspective view of the injector in a resting position before a first actuation and a second actuation, with the housing shown in phantom;

FIG. 4B is a perspective view of the injector in a deployed position after the first actuation and the second actuation, with the housing shown in phantom;

FIG. 5A is a side-elevational, cutaway view of a first chamber and the third chamber in fluid communication taken at 5A—5A of FIG. 3;

FIG. 5B is a side-elevational, cutaway view of a second chamber and the third chamber in fluid communication taken at 5B—5B of FIG. 3; and

FIG. 6 is a flowchart depicting a method for dispensing a first therapeutic agent and a second therapeutic agent.

### DETAILED DESCRIPTION

The following description of technology is merely exemplary in nature of the subject matter, manufacture and use of one or more inventions, and is not intended to limit the scope, application, or uses of any specific invention claimed in this application or in such other applications as may be filed claiming priority to this application, or patents issuing therefrom. Regarding methods disclosed, the order of the steps presented is exemplary in nature, and thus, the order of the steps can be different in various embodiments, including where certain steps can be simultaneously performed, unless expressly stated otherwise. “A” and “an” as used herein indicate “at least one” of the item is present; a plurality of such items may be present, when possible. Except where otherwise expressly indicated, all numerical quantities in this description are to be understood as modified by the word “about” and all geometric and spatial descriptors are to be understood as modified by the word “substantially” in describing the broadest scope of the technology. “About” when applied to numerical values indicates that the calculation or the measurement allows some slight imprecision in the value (with some approach to exactness in the value; approximately or reasonably close to the value; nearly). If, for some reason, the imprecision provided by “about” and/or “substantially” is not otherwise understood in the art with this ordinary meaning, then “about” and/or “substantially” as used herein indicates at least variations that may arise from ordinary methods of measuring or using such parameters.

All documents, including patents, patent applications, and scientific literature cited in this detailed description are incorporated herein by reference, unless otherwise expressly indicated. Where any conflict or ambiguity may exist between a document incorporated by reference and this detailed description, the present detailed description controls.

Although the open-ended term “comprising,” as a synonym of non-restrictive terms such as including, containing, or having, is used herein to describe and claim embodiments of the present technology, embodiments may alternatively be described using more limiting terms such as “consisting of” or “consisting essentially of.” Thus, for any given embodiment reciting materials, components, or process steps, the present technology also specifically includes embodiments consisting of, or consisting essentially of, such materials, components, or process steps excluding additional materials, components or processes (for consisting of) and excluding additional materials, components or processes affecting the significant properties of the embodiment (for consisting essentially of), even though such additional materials, components or processes are not explicitly recited in this application. For example, recitation of a composition or process reciting elements A, B and C specifically envisions embodiments consisting of, and consisting essentially of, A, B and C, excluding an element D that may be recited in the art, even though element D is not explicitly described as being excluded herein.

Disclosures of ranges are, unless specified otherwise, inclusive of endpoints and include all distinct values and further divided ranges within the entire range. Thus, for example, a range of “from A to B” or “from about A to about B” is inclusive of A and of B. Disclosure of values and ranges of values for specific parameters (such as amounts, weight percentages, etc.) are not exclusive of other values and ranges of values useful herein. It is envisioned that two or more specific exemplified values for a given parameter may define endpoints for a range of values that may be claimed for the parameter. For example, if Parameter X is exemplified herein to have value A and also exemplified to have value Z, it is envisioned that Parameter X may have a range of values from about A to about Z. Similarly, it is envisioned that disclosure of two or more ranges of values for a parameter (whether such ranges are nested, overlapping or distinct) subsume all possible combination of ranges for the value that might be claimed using endpoints of the disclosed ranges. For example, if Parameter X is exemplified herein to have values in the range of 1–10, or 2–9, or 3–8, it is also envisioned that Parameter X may have other ranges of values including 1–9, 1–8, 1–3, 1–2, 2–10, 2–8, 2–3, 3–10, 3–9, and so on.

When an element or layer is referred to as being “on,” “engaged to,” “connected to,” or “coupled to” another element or layer, it may be directly on, engaged, connected or coupled to the other element or layer, or intervening elements or layers may be present. In contrast, when an element is referred to as being “directly on,” “directly engaged to,” “directly connected to” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the example embodiments.

Spatially relative terms, such as “inner,” “outer,” “beneath,” “below,” “lower,” “above,” “upper,” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

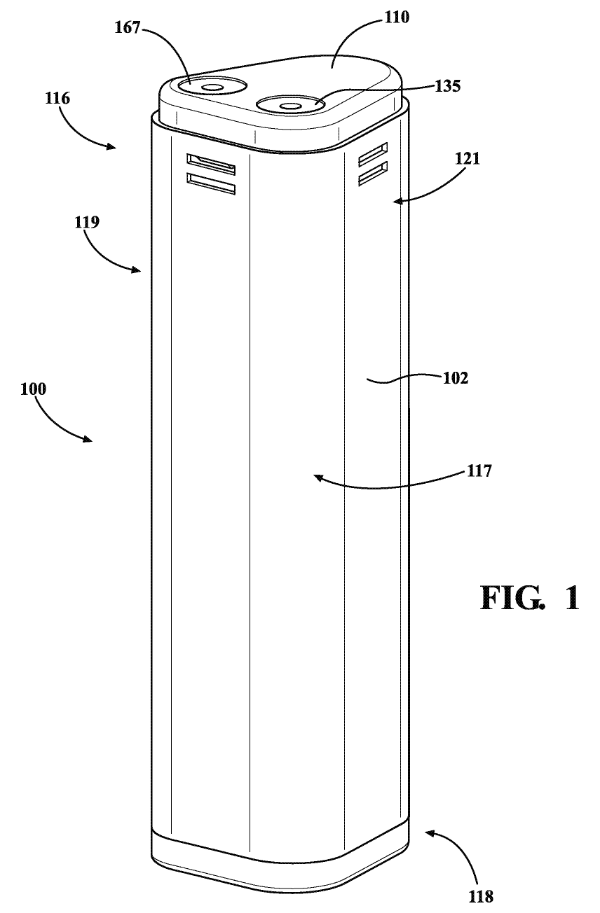

The present disclosure provides an injector 100 for a first therapeutic agent and a second therapeutic agent, aspects of which are shown generally in FIGS. 1-5B. The injector 100 can include a housing 102, a first chamber assembly 104, a second chamber assembly 106, a drive mechanism 108, and an actuator 110. The injector 100 can be configured as an autoinjector that can be self-administered by a patient. It should be appreciated that the autoinjector can offer an alternative to one or more syringes that are manually operated by the patient for administering one or more therapeutic agents. The autoinjector can be utilized by both trained professional and untrained users, such as the patient. A skilled artisan can select a suitable injector 100 type within the scope of the present disclosure. Certain aspects of the needle-based injector 100 described herein, for example, can include features and operability as described in PCT Application Publication No. 2024/108210, filed on November 20, 2023, and U.S. Provisional Patent Application No. 63/301,864 filed on January 21, 2022, the entire disclosures of which are incorporated herein by reference.

The first therapeutic agent and the second therapeutic agent can each include at least one of a therapeutic agent, a vaccine, a protein, a peptide, a gene, a compound or another pharmaceutically active ingredient. Examples of therapeutic agents suitable for use in the system include glucagon, insulin, adrenaline, epinephrine, anti-venom, atropine, antibody formulations, antidotes to chemical agents, and medications identified by tradenames including Acthar, Actimmune, Apokyn, AquaMephyton, Aranesp, Arixtra, Avonex, Betaseron, Bravelle, Butorphanol, Byetta, Calcijex, Calcitonin, Caverject, Cetrotide, Chorionic Gonadotropin, Cimzia, Copaxone, Copegus, DDAVP, D.H.E-45, Delatestryl, Delestrogen, Depo-Estradiol, Depo-Provera 150, Depo-SubQ Provera 104, Depo-Testosterone, Desmopressin, Dihydroergotamine, Edex, Eligard, Enbrel, Epipen, Epogen, Exjade, Faslodex, Fertinex, Follistim, Forteo, Fragmin, Fuzeon, Ganirelix acetate, Genotropin, Gleevec, Glucagon, Gonal, Heparin, Humatrope, Humira, Imitrex, Increlex, Infergen, Innohep, Insulin, Intron A, iPlex, Ketorolac, Kestrone, Kineret, Kuvan, Leukine, Leuprolide Acetate, Lovenox, Lupron, Luveris, Medroxyprogesterone, Menopur, Methotrexate, Miacalcin, Muse, Neumega, Neulasta, Neupogen, Nexavar, Norditropin, Novarel, Nutropin, Omnitrope, Orfadin, Ovidrel, Pegasys, Peg-Intron, Pregnyl, Procrit, Profasi, Progesterone, Pulmozyme, Raptiva, Rebetol, Rebif, Repronex, Revlimid, Ribasphere, Ribavirin, Saizen, Sandostatin, Sensipar, Serostim, Somatuline, Sprycel, Somavert, Stadol, Sumatriptan, Supprelin, Sutent, Symlin, Tarceva, Testosterone, Temodar, Tev-Tropin, Thalomid, Tobi, Tykerb, Vitamin B12, Vitamin D, Vitamin K, Xeloda, Zemplar, and Zorbtive.

The injector 100 can be capable of delivering a highly viscous formulation including the first therapeutic agent and the second therapeutic agent. Such formulations include those having a kinetic viscosity greater than that of water, having a kinetic viscosity greater than 30 centipoise, and having a kinetic viscosity between 30 centipoise and 1500 centipoise. These formulations can be used to deliver one or more biologicals, flowable tissues, connective tissue matrixes, and monoclonal antibodies. It should be appreciated that the first therapeutic agent and the second therapeutic agent can be the same, similar, or different depending on the specific needs of an intended user. A skilled artisan can select a suitable first therapeutic agent and second therapeutic agent within the scope of the present disclosure.

With reference to FIGS. 1-3, the injector can include the housing 102 that can define a majority of the outer structure the injector 100. The housing 102 can define a cavity 103 divided into at least three chambers, specially a first chamber 112, a second chamber 114, and a third chamber 115 that extend along a longitudinal axis (A) of the housing 102. The longitudinal axis (A) can extend from a proximal end 116 of the housing 102, where the actuator 110 is disposed, to a distal end 118 of the housing 102. The first chamber 112, the second chamber 114, and the third chamber 115 can be substantially parallel to one another and can extend substantially along the longitudinal axis (A) anywhere from a portion to an entire length of the housing 102.

The housing 102 can have a substantially triangular cross-section transverse to the longitudinal axis (A), including a triangular cross-section having rounded corners. The substantially triangular cross-section configuration can provide advantages over other configurations, particularly in terms of portability, ergonomics, and space efficiency. The compact triangular form factor can enable the injector 100 to be easily carried on the person, such as in a pocket, purse, or belt clip, without creating excessive bulk. Additionally, the triangular cross-section can facilitate storage within a medical kit, emergency response bags, or pharmaceutical packaging where space optimization and efficient stacking is desired.

The substantially triangular cross-section of the housing 102 can further relate to the internal design and operation of the injector 100. The first chamber 112 can be disposed within a first corner 117 of the housing 102, the second chamber 114 can be disposed within a second corner 119 of the housing 102, and the drive mechanism 108 can be disposed within a third corner 121 of the housing 102, with the corners being shown in FIG. 1. By positioning the first chamber 112 and the second chamber 114 side-by-side within respective corners of the triangular configuration, the housing 102 can achieve a space-efficient arrangement that minimizes the overall footprint of the injector 100. The arrangement allows both of the first chamber 112 and the second chamber 114 to be positioned adjacent to one another while maintaining separate, isolated fluid pathways for the first therapeutic agent and the second therapeutic agent, thereby militating against the risk of cross-contamination or chemical interaction during storage or operation. The drive mechanism 108 can therefore also be disposed alongside both the first chamber 112 and the second chamber 114 in the third chamber 115.

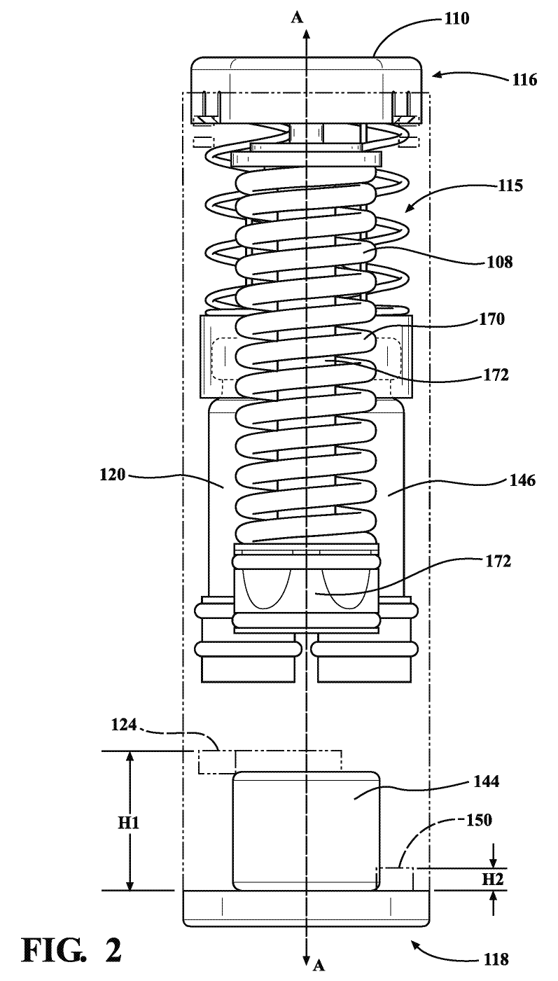

With reference to FIG. 2, the drive mechanism 108 can be disposed within the third corner of the housing 102. Positioning the drive mechanism 108 in the third corner can provide a balanced distribution of mass within the housing 102 and can facilitate a mechanical coupling between the drive mechanism 108 and both the first chamber 112 and the second chamber 114. In this way, the triangular configuration can allow a single, centralized drive mechanism 108 to actuate both the first chamber assembly 104 and the second chamber assembly 106 simultaneously or sequentially through a unified power source, militating against the need for redundant drive systems that can increase device complexity, cost, and potential points of failure.

The housing 102 can be formed from a rigid, biocompatible material suitable for medical device application, such as medical-grade plastic, polymer, or composite. Examples of suitable materials can include polycarbonate, acrylonitrile butadiene styrene (ABS), polyethylene, polypropylene, or combinations thereof. The housing 102 can be manufactured using injection molding, additive manufacturing, or other suitable fabrication techniques known in the art. A skilled artisan can select appropriate housing materials and fabrication methods within the scope of the present disclosure. The housing 102 can further include one or more sidewalls that define an exterior surface of the triangular cross-section and enclose the internal components of the injector 100. The sidewalls can provide structural support and protection for the first chamber assembly 104, the second chamber assembly 106, and the drive mechanism 108. The housing 102 can be configured to maintain the sterility of the internal components prior to use and can include seals, caps, or protective covers at the proximal and distal ends to militate against contamination.

The housing 102 can be constructed to provide sufficient structural rigidity and mechanical strength to withstand various environmental factors encountered during storage, transport, and use. The rigid construction can protect the internal components from mechanical shock, impact forces, compression, and other physical stresses that can occur during handling or accidental dropping of the device. The housing 102 material can be selected to maintain structural integrity across a range of temperatures, including typical storage conditions (e.g., 2°C to 25°C or 36°F to 77°F) as well as temporary exposure to elevated or reduced temperatures that can be encountered during shipping or field use. The housing 102 can further be resistant to moisture ingress, chemical exposure, and ultraviolet radiation degradation, ensuring that the device remains functional throughout its intended shelf life.

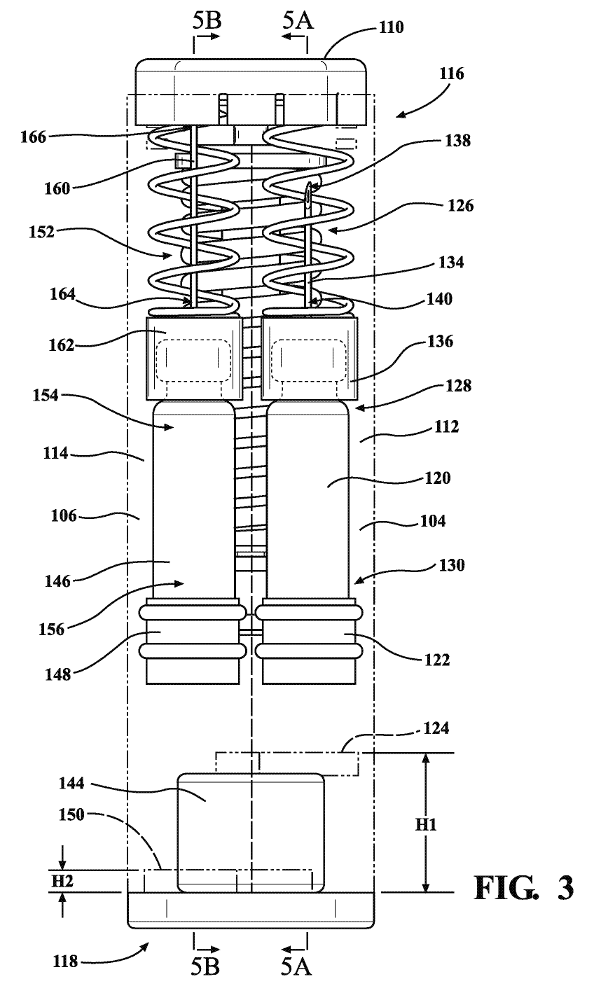

Turning first to the mechanical movement within the first chamber assembly 104 and the second chamber assembly 106 during operation, FIG. 3 depicts the injector 100 including the first chamber assembly 104. The first chamber assembly 104 can be disposed within the first chamber 112 and can include a first container 120, a first plunger 122, a first channel 124, and a first needle assembly 126. The first chamber assembly 104 can function as an integrated system for storing, actuating, and delivering the first therapeutic agent to a patient.

The first container 120 can be configured to contain and house the first therapeutic agent prior to delivery. The first container 120 can have an elongated, cylindrical body with a substantially circular cross-section when viewed along the longitudinal axis (A). The cylindrical configuration can provide uniform internal volume for consistent dosing and facilitates smooth translation within the first chamber 112 during actuation. The first container 120 can include a proximal end 128 and a distal end 130, with the proximal end 128 configured to interface with the first needle assembly 126 during operation.

The first container 120 can include a first septum 132 disposed closer to the proximal end 116 of the housing 102 than the distal end 118 of the housing 102. The first septum 132 can function as a sterile barrier that seals the first therapeutic agent within the first container 120 and militates against contamination from the external environment during storage. The first septum 132 can be formed from an elastomeric material, such as butyl rubber, silicone rubber, or a thermoplastic elastomer, for example, that is capable of being pierced by a first needle 134 while maintaining a fluid-tight seal prior to piercing. The first septum 132 can be secured to the first container 120 using a crimp seal, adhesive bonding, or other suitable attachment known in the pharmaceutical packaging.

The first plunger 122 can be disposed within the first chamber 112 at a position proximal to the first container 120. When activated by the hydraulic fluid being pushed into the first chamber 112 by the drive mechanism 108, the first plunger 122 can contact the distal end 130 of the first container 120 and perform three functions in sequence. First, the first plunger 122 can drive the first container 120 upward into a first cap 136, positioning the first septum 132 to align with the first needle 134 where a distal end 140 of the first needle 134 punctures the first septum 132 to establish initial fluid communication between the first container 120 and the first needle 134. Second, the continued upward movement of the first plunger 122 can advance the entire first needle assembly 126 proximally toward the patient, allowing a proximal end 138 of the first needle 134 to puncture through a first membrane 135 on the housing 102 and penetrate the tissue of the patient. Third, once the first needle 134 has successfully punctured the first membrane 135 and entered the patient, the first plunger 122 can continue advancement within the first container 120, applying hydraulic pressure to drive the first therapeutic agent through the first needle 134 and deliver the first therapeutic agent into the patient. The sequential three-stage operation including first container 120 positioning, first needle 134 deployment, and first therapeutic agent delivery can be powered by the drive mechanism 108 actuated upon activation of the actuator 110.

The first plunger 122 can have a substantially circular cross-section corresponding to the diameter of the first chamber 112, allowing the first plunger 122 to translate smoothly along the longitudinal axis (A) while maintaining alignment with the first container 120. As described herein, the first plunger 122 can be operatively associated with the first container 120 such that upward movement of the first plunger 122 along the longitudinal axis (A) causes corresponding upward movement of the first container 120. The first plunger 122 can be formed from a rigid or semi-rigid material capable of transmitting force from the drive mechanism 108 to the first container 120 without deformation. Suitable materials can include medical-grade plastics, metals, or composite materials, for example.

The first needle assembly 126 can include the first needle 134 and a first cap 136. The first needle 134 can be coupled to the first cap 136 to militate against the first needle 134 from fully exiting or falling out of the housing 102 through the first membrane 135 in operation. The first needle assembly 126 can be positioned at or near the proximal end 116 of the housing 102 to facilitate exit of the first needle 134 through the first membrane 135 in operation.

The first needle 134 can include a dual-ended needle having a proximal end 138 and a distal end 140, with both the proximal end 138 and the distal end 140 being sharpened to facilitate piercing. The first needle 134 can include a hollow bore or lumen extending through an entire length of the first needle 134, providing a continuous fluid pathway for the first therapeutic agent. The proximal end 138 of the first needle 134 can be oriented toward the proximal end 116 of the housing 102 and the first membrane 135. The distal end 140 of the first needle 134 can be positioned to receive and pierce the first septum 132 of the first container 120 as the first container 120 advances during actuation.

The first cap 136 can be fixedly positioned within the first chamber 112 closer to the proximal end 116 of the housing 102 than the distal end 118 of the housing 102 and can be coupled to the first needle 134. The first cap 136 can support and retain the first needle 134 in a predetermined orientation relative to the first chamber 112 and the longitudinal axis (A). The first cap 136 can define a first interior cavity 142 configured to receive the first container 120 as the first container 120 advances upward during operation. The first cap 136 can be secured to the first needle 134 in a fixed position such that the distal end 140 of the first needle 134 remains stationary as the first container 120 moves toward the first needle 134.

It should be appreciated that the second chamber assembly 106 can be configured and can function similarly to the first chamber assembly 104. As shown in FIG. 3, the injector 100 can include the second chamber assembly 106. The second chamber assembly 106 can be disposed within the second chamber 114 and can include a second container 146, a second plunger 148, a second channel 150, and a second needle assembly 152. The second chamber assembly 106 can function as an integrated system for storing, actuating, and delivering the second therapeutic agent to a patient.

The second container 146 can be configured to contain and house the second therapeutic agent prior to delivery. The second container 146 can have an elongated, cylindrical body with a substantially circular cross-section when viewed along the longitudinal axis (A). The cylindrical configuration can provide uniform internal volume for consistent dosing and facilitates smooth translation within the second chamber 114 during actuation. The second container 146 can include a proximal end 154 and a distal end 156, with the proximal end 154 configured to interface with the second needle assembly 152 during operation.

The second container 146 can include a second septum 158 disposed closer to the proximal end 116 of the housing 102 than the distal end 118 of the housing 102. The second septum 158 can function as a sterile barrier that seals the second therapeutic agent within the second container 146 and militates against contamination from the external environment during storage. The second septum 158 can be formed from an elastomeric material, such as butyl rubber, silicone rubber, or a thermoplastic elastomer, for example, that is capable of being pierced by a second needle 160 while maintaining a fluid-tight seal prior to piercing. The second septum 158 can be secured to the second container 146 using a crimp seal, adhesive bonding, or other suitable attachment known in the pharmaceutical packaging.

The second plunger 148 can be disposed within the second chamber 114 at a position proximal to the second container 146. When activated by the hydraulic fluid being pushed into the second chamber 114 by the drive mechanism 108, the second plunger 148 can contact the distal end 156 of the second container 146 and perform three functions in sequence. First, the second plunger 148 can drive the second container 146 upward into a second cap 162, positioning the second septum 158 to align with the second needle 160 where a distal end 164 of the second needle 160 punctures the second septum 158 to establish initial fluid communication between the second container 146 and the second needle 160. Second, the continued upward movement of the second plunger 148 can advance the entire second needle assembly 152 proximally toward the patient, allowing a proximal end 166 of the second needle 160 to puncture through a second membrane 167 on the housing 102 and penetrate the tissue of the patient. Third, once the second needle 160 has successfully punctured the second membrane 167 and entered the patient, the second plunger 148 can continue advancement within the second container 146, applying hydraulic pressure to drive the second therapeutic agent through the second needle 160 and deliver the second therapeutic agent into the patient. The sequential three-stage operation including second container 146 positioning, second needle 160 deployment, and second therapeutic agent delivery can be powered by the drive mechanism 108 actuated upon activation of the actuator 110.

The second plunger 148 can have a substantially circular cross-section corresponding to the diameter of the second chamber 114, allowing the second plunger 148 to translate smoothly along the longitudinal axis (A) while maintaining alignment with the second container 146. As described herein, the second plunger 148 can be operatively associated with the second container 146 such that upward movement of the second plunger 148 along the longitudinal axis (A) causes corresponding upward movement of the second container 146. The second plunger 148 can be formed from a rigid or semi-rigid material capable of transmitting force from the drive mechanism 108 to the second container 146 without deformation. Suitable materials can include medical-grade plastics, metals, or composite materials, for example.

The second needle assembly 152 can include the second needle 160 and a second cap 162. The second needle 160 can be coupled to the second cap 162 to militate against the second needle 160 from fully exiting or falling out of the housing 102 through the second membrane 167 in operation. The second needle assembly 152 can be positioned at or near the proximal end 116 of the housing 102 to facilitate exit of the second needle 160 through the second membrane 167 in operation.

The second needle 160 can include a dual-ended needle having a proximal end 166 and a distal end 164, with both the proximal end 166 and the distal end 164 being sharpened to facilitate piercing. The second needle 160 can include a hollow bore or lumen extending through an entire length of the second needle 160, providing a continuous fluid pathway for the second therapeutic agent. The proximal end 166 of the second needle 160 can be oriented toward the proximal end 116 of the housing 102 and the second membrane 167. The distal end 164 of the second needle 160 can be positioned to receive and pierce the second septum 158 of the second container 146 as the second container 146 advances during actuation.

The second cap 162 can be fixedly positioned within the second chamber 114 closer to the proximal end 116 of the housing 102 than the distal end 118 of the housing 102 and can be coupled to the second needle 160. The second cap 162 can support and retain the second needle 160 in a predetermined orientation relative to the second chamber 114 and the longitudinal axis (A). The second cap 162 can define a second interior cavity 143 configured to receive the second container 146 as the second container 146 advances upward during operation. The second cap 162 can be secured to the second needle 160 in a fixed position such that the distal end 164 of the second needle 160 remains stationary as the second container 146 moves toward the second needle 160.

Turning now to the drive mechanism 108, FIG. 2 depicts a spring-powered hydraulic system 168 that can combine the reliability of a spring with the flexibility and control of hydraulics. For example, the actuator 110 can include a button, a tab, a slider, a knob, or other mechanism coupled to the drive mechanism 108, and upon activation of the actuator 110, the drive mechanism 108 can be configured to deliver stored energy to drive the delivery of the therapeutic agents. A skilled artisan can select a suitable actuator 110 within the scope of the present disclosure. The drive mechanism 108 can include a deployment spring 170 that drives a piston 172, which can then deliver hydraulic fluid to move the first plunger 122 within the first chamber 112 and the second plunger 148 within the second chamber 114. The deployment spring 170 can be locked in a compressed configuration and released upon actuation of the actuator 110. The hydraulic system 168 can power both the first chamber assembly 104 and the second chamber assembly 106 through a single integrated power source, maintaining high reliability while reducing potential points of failure compared to systems utilizing multiple springs, gas canisters, or electronic systems.

With reference to FIG. 2, the drive mechanism 108 can include a barrier 144 positioned within the housing 102 near the distal end 118 to selectively control hydraulic fluid flow between the first chamber 112 and the second chamber 114. The barrier 144 is configured as a frangible seal that initially blocks the hydraulic fluid pathway to the second chamber 114, thereby directing fluid through the first channel 124 during initial actuation. Upon subsequent actuation generating sufficient hydraulic pressure, the barrier 144 can rupture to open the second channel 150 to the second chamber 114 when under a predetermined pressure. The barrier 144 can be configured as a unitary body with controlled failure characteristics, as shown in FIG. 2. Suitable materials for the barrier 144 include thin-walled or scored sections of engineering plastics such as polycarbonate, acrylonitrile butadiene styrene (ABS), polyoxymethylene (POM), polyethylene, or polymer films configured to rupture at predetermined pressure thresholds, for example.

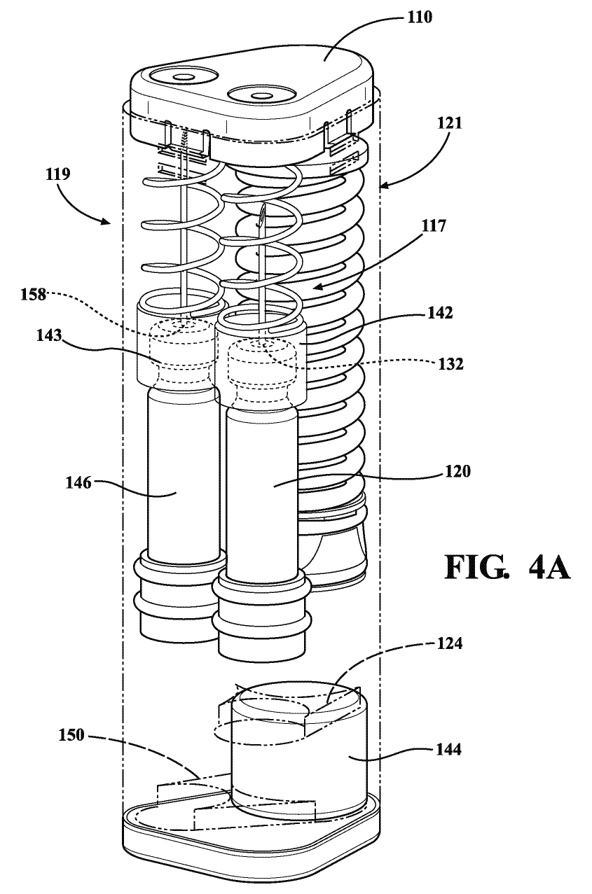

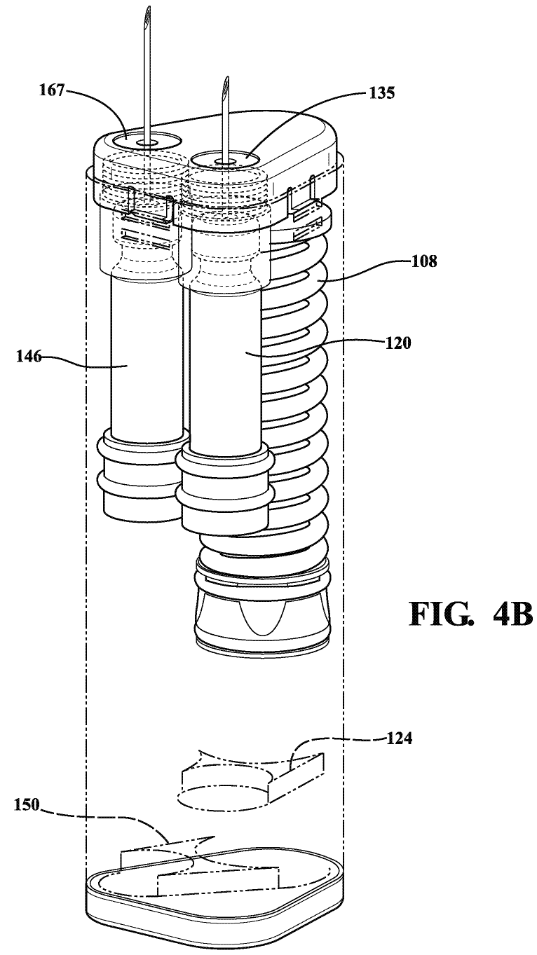

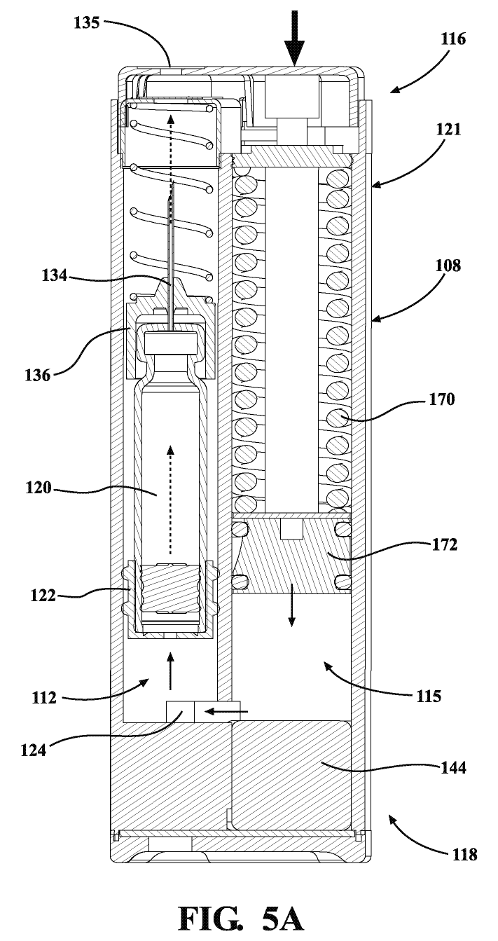

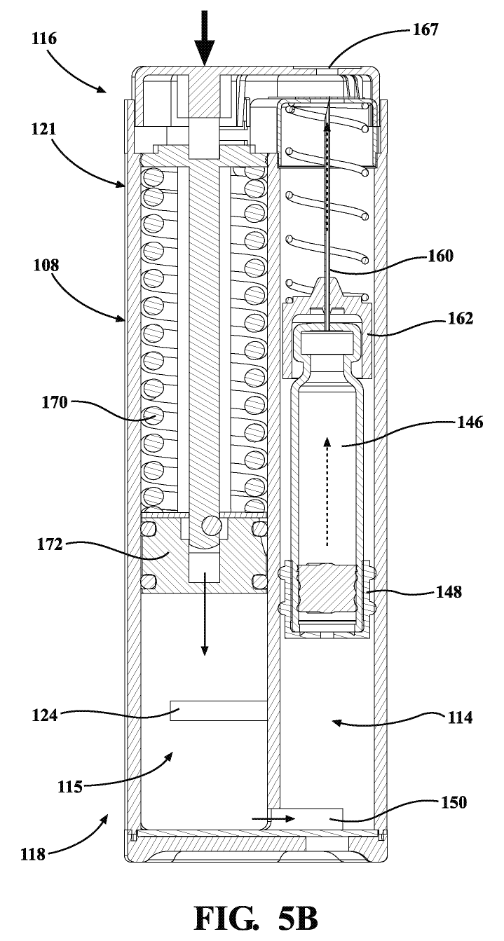

With reference to FIGS. 5A and 5B, the first channel 124 of the first chamber assembly 104 and the second channel 150 of the second chamber assembly 106 can be coupled to the housing 102 in stationary positions at different heights within the first chamber 112 and the second chamber 114. The first channel 124 and the second channel 150 can each operate as a passageway between the third chamber 115 housing the drive mechanism 108 and the first chamber 112 and the second chamber 114, respectively. With reference to FIG. 3, the first channel 124 can be positioned closer to the proximal end 116 of the housing 102 than the second channel 150 and can permit for fluid communication between the third chamber 115 housing the drive mechanism 108 and the first chamber 112. With further reference to FIG. 3, the second channel 150 can permit for fluid communication between the third chamber 115 housing the drive mechanism 108 and the second chamber 114. As shown in FIG. 4A, the second channel 150 can initially be blocked by the barrier 144 to militate against hydraulic fluid entering the second chamber 114 upon the first activation of the actuator 110. Each of the first channel 124 and the second channel 150 can be configured as a fluid conduit that directs hydraulic fluid into the first chamber 112 and the second chamber 114, respectively, to drive movement of the first container 120 and the second container 146. In certain embodiments, the first channel 124 and second channel 150 can be integrally molded or machined as part of the housing 102, forming integrated fluid ports. In an alternative embodiment, the first channel 124 and the second channel 150 can be separately fabricated components that are coupled to the housing 102 through mechanical fasteners, adhesive bonding, welding, press-fitting, threaded connections, or combinations thereof.

The first channel 124 and the second channel 150 can be configured as a port, passage, aperture, conduit, orifice, or similar fluid delivery feature within the housing 102 that opens into the first chamber 112 and the second chamber 114, respectively. The first channel 124 and the second channel 150 can have various cross-sectional geometries including rectangular, circular, annular, slotted, or irregular shapes depending on the desired fluid flow characteristics and delivery rate into the respective chambers. An outlet surface of the first channel 124 and the second channel 150, through which hydraulic fluid is discharged into the first chamber 112 and the second chamber 114, can be smooth, beveled, chamfered, or contoured to control the direction and distribution of fluid flow during actuation.

With reference to FIG. 2, the first channel 124 can be positioned at a first height (H1) within the first chamber 112 and can be operatively associated with the first plunger 122. The second channel 150 can be positioned at a second height (H2) within the second chamber 114 and can be operatively associated with the second plunger 148. The positioning within the respective first chamber 112 and the second chamber 114 can mean that the first channel 124 and the second plunger 148 extend into or are aligned with the first chamber 112 and the second chamber 114 such that the first channel 124 and the second channel 150 can permit for fluid engagement between the hydraulic fluid and the first plunger 122 and the second plunger 148, respectively.

As described herein, the second height (H2) can be different from the first height (H1) such that the first channel 124 and the second channel 150 can be positioned at different elevations along the longitudinal axis (A) of the housing 102. The different heights of the first channel 124 and the second channel 150 positioned with the first chamber 112 and the second chamber 114 can be selected to optimize fluid delivery into the first chamber 112 and the second chamber 114, respectively, to effectively drive movement of the first container 120 and the second container 146. The sequential delivery of the first therapeutic agent and the second therapeutic agent can be controlled by the selective rupture of the barrier 144, which can initially direct hydraulic fluid through the first channel 124 during a first actuation, and subsequently allows hydraulic fluid to flow through the second channel 150 during a second actuation after the barrier 144 ruptures. The timing interval between drug deliveries can be determined by the user through manual control of successive actuations of the drive mechanism 108 or can be predetermined based on the pressure threshold required to rupture the barrier 144 during continued actuation.

In certain embodiments, the spacing between the first channel 124 and second channel 150 can range from about 0.5 millimeters to about 50 millimeters, from about 1 millimeter to about 30 millimeters, from about 2 millimeters to about 20 millimeters, or from about 5 millimeters to about 15 millimeters. The specific spacing selected depends on factors including the velocity of barrier travel (which is influenced by the spring force and throttle settings), the desired time delay between injections, the therapeutic requirements of the drug combination being delivered, and the physical dimensions of the housing 102, the first chamber 112, and the second chamber 114.

In operation, first actuation of the drive mechanism 108 can generate hydraulic pressure that drives hydraulic fluid from the third chamber 115 through the first channel 124 and into the first chamber 112. The hydraulic fluid can preferentially flow through the first channel 124 rather than the second channel 150 for multiple reasons. First, the first channel 124 can be positioned closer to the drive mechanism 108, creating a shorter fluid pathway with lower hydraulic resistance. Second, the barrier 144 can initially block or seal the hydraulic fluid pathway leading to the second channel 150, thereby militating against fluid flow to the second chamber 114 and making the first channel 124 the path of least resistance. The combination of proximity and pathway availability can ensure that hydraulic fluid is directed exclusively or predominantly through the first channel 124 during the first actuation.

With reference to FIG. 5A in which the hydraulic fluid path is depicted with a solid arrow path and the movement path of the first chamber 112 is depicted with a dashed arrow path, the hydraulic fluid discharged through the first channel 124 can enter the first chamber 112 and accumulate beneath the first container 120. As hydraulic fluid continues to flow into the first chamber 112, hydraulic pressure can build and exert an upward force on the first container 120. The hydraulic pressure can overcome any frictional forces, sealing forces, or resistance within the first chamber 112 and can drive the first container 120 upward along the longitudinal axis (A) toward the first cap 136, as described herein. As the first container 120 moves upward, the first needle 134 can puncture the first septum 132 and the first membrane 135, creating a fluid pathway for the first therapeutic agent to be delivered from the first container 120, through the first needle 134, and into the target tissue or administration site.

With reference to FIG. 5B and during the second actuation of the drive mechanism 108, additional hydraulic pressure can be generated by the drive mechanism 108. The increased hydraulic pressure can be directed toward the barrier 144, which can continue to block the second channel 150. As the hydraulic pressure increases and reaches or exceeds a predetermined threshold pressure, the barrier 144 can rupture, break, burst, or otherwise fail in a controlled manner. The rupture of the barrier 144 can open the previously blocked second channel 150, allowing hydraulic fluid to flow into the second chamber 114. As described herein, the barrier 144 can be formed of a material having wall thicknesses, scored sections, or geometric features that control the pressure at which rupture occurs, thereby providing predictable and reliable sequential operation.

Once the barrier 144 ruptures and the second channel 150 is opened, hydraulic fluid can flow through the second channel 150 and enter the second chamber 114, as shown in FIG. 5B in which the hydraulic fluid path is depicted with a solid arrow path and the movement path of the second chamber 114 is depicted with a dashed arrow path. The hydraulic fluid can accumulate beneath the second container 146, creating hydraulic pressure that exerts an upward force on the second container 146. The hydraulic pressure can drive the second container 146 upward along the longitudinal axis (A), causing the second needle 160 secured to or extending from the second container 146 to pierce the second septum 158 and the second membrane 167. The piercing of the second septum 158 can create a fluid pathway for the second therapeutic agent to be delivered from the second container 146, through the second needle 160, and into the target tissue or administration site, completing the sequential delivery of both therapeutic agents.

The timing interval between the delivery of the first therapeutic agent and the second therapeutic agent can be controlled in various ways. In certain embodiments, the timing can be determined by the user through manual control of successive actuations of the drive mechanism 108, allowing the user to complete the first actuation, wait a desired period of time, and then perform the second actuation to trigger barrier 144 rupture and second drug delivery. In an alternative embodiment, the timing can be predetermined or influenced by the pressure threshold required to rupture the barrier 144, such that continuous or rapid successive actuation can result in shorter intervals between deliveries, while slower or gentler actuation can result in longer intervals. The barrier rupture pressure threshold can be engineered to provide consistent timing characteristics across multiple uses of the device.

It should be appreciated that the hydraulic drive mechanism 108 of the injector 100 can be optimized for the delivery of high-volume and highly viscous therapeutic agents. The spring-powered hydraulic drive mechanism 108 can provide sustained, controlled pressure throughout the entire delivery cycle, enabling complete extrusion of large therapeutic volume without the pressure decay or force limitations that can occur with purely spring-driven systems.

The drive mechanism 108 can accommodate a therapeutic agent having viscosities substantially greater than water, including biologics, flowable tissues, connective tissue matrices, and monoclonal antibodies with kinetic viscosities exceeding 30 centipoise and ranging up to 1500 centipoise or higher. The drive mechanism 108 can generate sufficient hydraulic pressure to overcome the flow resistance created by viscous therapeutic agents passing through the restricted inner diameter of the first needle 134 and the second needle 160, while simultaneously controlling the rate of pressure application to militate against container rupture, septum deformation, or needle clogging. The hydraulic fluid transfers force from the deployment spring 170 to the first plunger 122 and the second plunger 148 in a manner that distributes pressure evenly across the therapeutic agent containers, reducing localized stress concentrations that could damage glass cartridges or cause mechanical failure.

In certain embodiments, the drive mechanism 108 can include throttle control features that regulate the flow rate of the hydraulic fluid to the first chamber 112 and the second chamber 114 independently. The throttle control can be configured as orifices, channels, or adjustable flow restrictors positioned between the piston 172 and the respective chambers, allowing for tailored pressure profiles that accommodate different viscosity requirements for the first therapeutic agent and the second therapeutic agent. For example, where the first therapeutic agent includes a highly viscous monoclonal antibody formulation and the second therapeutic agent includes a lower viscosity small molecule solution, the throttle associated with the first chamber 112 can be configured with a larger orifice diameter or reduced flow restriction to permit higher flow rates and greater pressure transmission, while the throttle associated with the second chamber 114 can be configured with more restrictive flow characteristics. The independent throttle control ensures that each therapeutic agent is delivered at an optimal rate regardless of viscosity differences, maintaining dosing accuracy and preventing incomplete delivery.

Returning now to the first needle 134 and the second needle 160, which can each be independently configured for intramuscular (IM) or subcutaneous (SC) delivery of the first therapeutic agent and the second therapeutic agent. As described herein, both the first needle 134 and the second needle 160 can be double-ended, with a sharp first end configured to penetrate tissue for patient administration and sharp second end for puncturing through the septum of the respective drug containers to access the therapeutic agents.

The integration of dual needle functionality for IM/SC delivery can be advantageous in clinical scenarios requiring multiple drugs for effective treatment. The injector 100 can militate against chemical or physical interactions between therapeutic agents during storage and delivery by isolating each therapeutic agent from the other. This separation ensures that drugs requiring different storage conditions or exhibiting incompatibility issues can be safely co-delivered without cross-contamination at the injection site.

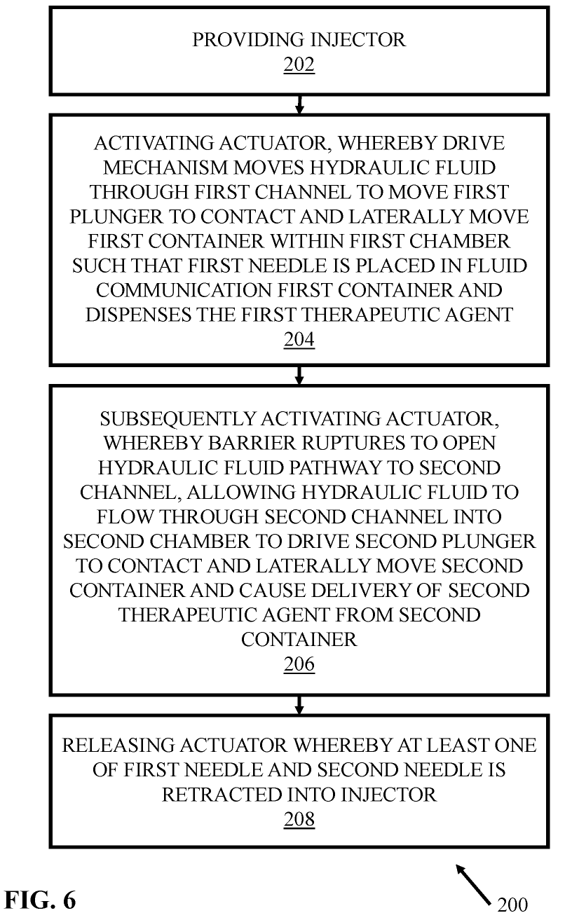

The present disclosure further provides a method 200 for dispensing a first therapeutic agent and a second therapeutic agent, shown generally in FIG. 6. The method 200 can include a step 202 of providing the injector 100 as described herein. In a step 204, the method 200 can including activating the actuator 110, whereby the drive mechanism 108 moves hydraulic fluid through the first channel 124 to move the first plunger 122 to contact and laterally move the first container 120 within the first chamber 112 such that the first needle 134 is placed in fluid communication the first container 120 and dispenses the first therapeutic agent.

The method can further include a step 206 of subsequently activating the actuator 110, whereby the barrier 144 ruptures to open the hydraulic fluid pathway to the second channel 150, allowing hydraulic fluid to flow through the second channel 150 into the second chamber 114 to drive the second plunger 148 to contact and laterally move the second container 146 and cause delivery of the second therapeutic agent from the second container 146. The actuator 110 can be released in a step 208 causing at least one of the first needle 134 and the second needle 160 to be retracted into the injector 100.

Example embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms, and that neither should be construed to limit the scope of the disclosure. In some example embodiments, well-known processes, well-known device structures, and well-known technologies are not described in detail. Equivalent changes, modifications and variations of some embodiments, materials, compositions and methods can be made within the scope of the present technology, with substantially similar results.

What is claimed is:

1. An injector for delivering a first therapeutic agent and a second therapeutic agent to a patient, comprising:
a housing defining a first chamber, a second chamber, and a third chamber and having a longitudinal axis;
a drive mechanism disposed within the third chamber and including a hydraulic fluid and a barrier configured to rupture under hydraulic pressure at a predetermined pressure;
an actuator operatively coupled to the drive mechanism;
a first chamber assembly disposed within the first chamber and including
a first container disposed within the first chamber and configured to house the first therapeutic agent,
a first plunger operatively associated with the first container, and
a first channel coupled to the housing at a first height within the first chamber and configured to direct the hydraulic fluid into the first chamber; and
a second chamber assembly disposed within the second chamber and including
a second container disposed within the second chamber and configured to house the second therapeutic agent,
a second plunger operatively associated with the second container, and
a second channel coupled to the housing at a second height within the second chamber and configured to direct the hydraulic fluid into the second chamber, wherein the second height is different from the first height,
wherein:
the first channel is positioned closer to the drive mechanism than the second channel along the longitudinal axis, the barrier blocking a hydraulic fluid pathway to the second channel,
upon a first actuation of the actuator, the hydraulic fluid flows through the first channel into the first chamber to drive the first container and deliver the first therapeutic agent from the first container, and
upon a second actuation of the actuator, the barrier ruptures to open the hydraulic fluid pathway to the second channel, allowing the hydraulic fluid to flow through the second channel into the second chamber to drive the second container and cause delivery of the second therapeutic agent from the second container.

2. The injector of claim 1, wherein the first chamber assembly further includes a first needle assembly having a first needle and a first cap, the first needle disposed within the first cap.

3. The injector of claim 2, wherein the first needle is a dual-ended needle having a first end and a second end, both the first end and the second end being sharpened.

4. The injector of claim 3, wherein movement of the first plunger causes the first container to advance such that a first end of the first needle punctures a first septum of the first container to establish fluid communication between the first container and the patient.

5. The injector of claim 2, wherein the first cap defines a first interior cavity configured to receive the first container as the first container advances during operation.

6. The injector of claim 1, wherein the second chamber assembly further includes a second needle assembly having a second needle and a second cap, the second needle disposed within the second cap.

7. The injector of claim 6, wherein movement of the second plunger causes the second container to advance such that a first end of the second needle punctures a second septum of the second container to establish fluid communication between the second container and the patient.

8. The injector of claim 1, wherein the drive mechanism includes a spring-powered hydraulic system.

9. The injector of claim 8, wherein the spring-powered hydraulic system includes a deployment spring, a piston, and the hydraulic fluid.

10. The injector of claim 9, wherein the piston is driven by the deployment spring to deliver the hydraulic fluid to move at least one of the first plunger and the second plunger.

11. The injector of claim 1, wherein the first channel and the second channel are integrally molded with the housing.

12. The injector of claim 1, wherein the barrier includes a member selected from a group consisting of polycarbonate, acrylonitrile butadiene styrene (ABS), polyoxymethylene (POM), polyethylene, and combinations thereof.

13. The injector of claim 1, wherein a first needle of the first chamber assembly is configured for subcutaneous delivery and a second needle of the second chamber assembly is configured for intramuscular delivery.

14. The injector of claim 1, wherein the first chamber and the second chamber are substantially parallel to one another and extend substantially along a majority of a length of the longitudinal axis.

15. The injector of claim 1, wherein the first plunger directly abuts a distal end of the first container.

16. The injector of claim 15, wherein the second plunger directly abuts a distal end of the second container.

17. The injector of claim 1, wherein the first chamber, the second chamber, and the third chamber are substantially parallel to one another and extend substantially along a majority of a length of the longitudinal axis.

18. A method for dispensing a first therapeutic agent and a second therapeutic agent, comprising:
providing an injector for a first therapeutic agent and a second therapeutic agent including
a housing defining a first chamber, a second chamber, and a third chamber and having a longitudinal axis,
a drive mechanism disposed within the third chamber and including a hydraulic fluid and a barrier configured to rupture under hydraulic pressure at a predetermined pressure,
an actuator operatively coupled to the drive mechanism,
a first chamber assembly disposed within the first chamber and including
a first container disposed within the first chamber and configured to house the first therapeutic agent,
a first plunger operatively associated with the first container, and
a first channel coupled to the housing at a first height within the first chamber and configured to direct the hydraulic fluid into the first chamber, and
a second chamber assembly disposed within the second chamber and including
a second container disposed within the second chamber and configured to house the second therapeutic agent,
a second plunger operatively associated with the second container, and
a second channel coupled to the housing at a second height within the second chamber and configured to direct the hydraulic fluid into the second chamber, wherein the second height is different from the first height,
wherein:
the first channel is positioned closer to the drive mechanism than the second channel along the longitudinal axis, and wherein the barrier initially blocks a hydraulic fluid pathway to the second channel,
upon a first actuation of the actuator, the hydraulic fluid flows through the first channel into the first chamber to drive the first container and cause delivery of the first therapeutic agent from the first container, and
upon a second actuation of the actuator, the barrier ruptures to open the hydraulic fluid pathway to the second channel, allowing the hydraulic fluid to flow through the second channel into the second chamber to drive the second container and cause delivery of the second therapeutic agent from the second container; and
activating the actuator, whereby the drive mechanism moves the hydraulic fluid through the first channel to move the first plunger to contact and laterally move the first container within the first chamber such that a first needle is placed in fluid communication the first container and dispenses the first therapeutic agent.

19. The method of claim 18, further including a step of subsequently activating the actuator, whereby the barrier ruptures to open the hydraulic fluid pathway to the second channel, allowing the hydraulic fluid to flow through the second channel into the second chamber to drive the second plunger to contact and laterally move the second container and cause delivery of the second therapeutic agent from the second container.

20. The method of claim 19, further including a step of releasing the actuator whereby at least one of the first needle and the second needle is retracted into the injector.
